(19) United States
(12) Patent Application Publication
(10) Pub. No.: US 2022/0211953 A1
(43) Pub. Date: Jul. 7, 2022

(54) FINE NEEDLE INJECTION THERAPY DEVICE

(71) Applicant: Mayo Foundation for Medical Education and Research, Rochester, MN (US)

(72) Inventors: Michael J. Levy, Rochester, MN (US); Ferga C. Gleeson, Rochester, MN (US); Louis-Michel Wong Kee Song, Rochester, MN (US)

(21) Appl. No.: 17/593,507

(22) PCT Filed: Mar. 20, 2020
(86) PCT No.: PCT/US2020/023784

§ 371 Date: Sep. 20, 2021

Related U.S. Application Data
(60) Provisional application No. 62/821,717, filed on Mar. 21, 2019.

(51) Int. Cl. A61M 5/32; A61L 24/06; A61B 17/00; A61M 5/00; A61B 17/12

(52) U.S. Cl. CPC A61M 5/3294; A61M 5/329; A61L 24/06; A61B 2017/1205; A61M 5/007; A61B 17/1215; A61M 2205/0238; A61B 17/00491

(57) ABSTRACT

This document describes methods and materials for improving cancer treatment. For example, this document describes methods and devices for local tumor control and customizable patient treatment.

### CROSS-REFERENCE TO RELATED APPLICATIONS

This application claims the benefit of U.S. Provisional Application Ser. No. 62/821,717, filed Mar. 21, 2019. The disclosure of the prior application is considered part of (and is incorporated by reference in) the disclosure of this application.

### BACKGROUND

### 1. Technical Field

This document relates to methods and materials for improving cancer treatment. For example, this document relates to methods and devices for local tumor control and customizable patient treatment.

### 2. Background Information

Chemotherapy and radiation therapy have been used to treat pancreatic ductal adenocarcinoma (PDAC). Despite the introduction of many new agents touted to transform care and outcomes, the median survival expectancy for locally advanced pancreatic cancer (LAPC) remains at only 9-11 months, even with the administration of chemoradiotherapy, and only at 6 months for patients with metastatic disease. Neoadjuvant therapy is routinely administered to patients with LAPC with the aim of tumor downstaging to potentially allow subsequent resection. Unfortunately, for patients who have unresectable tumors, excluding borderline resectable status, chemoradiotherapy seldom leads to tumor downstaging and a complete resection with negative margins. Prolonged survival is rare. A great challenge to the delivery of systemic chemotherapy is the suboptimal intratumoral penetration of therapy that occurs secondary to a hostile tumor microenvironment that is partly attributed to tumor-induced stromal desmoplasia. Therefore, despite recent advances, chemoradiation serves only a palliative role in treatment of most patients with PDAC.

The limited efficacy of conventional PDAC therapy results from the tumor biology, severe desmoplasia and altered tumor-induced biophysical properties that result in neoplastic islands interspersed among dense stroma. This resulting tumor structure topography alters the pattern and limits chemotherapy penetrance to the tumor.

Many of the biological and technical limitations apply to other solid pancreatic tumors (e.g., functioning and non-functioning neuroendocrine tumors, lymphoma, secondary metastasis to the pancreas, etc.). There are also several cystic pancreatic tumors (CPT) such as intraductal papillary mucinous neoplasia, mucinous cystic neoplasia, and serous cystadenomas, among others that experience the biological and technical limitations.

### SUMMARY

This document describes methods and materials for improving the delivery of cancer treatment. For example, this document describes methods and devices for local tumor control and customizable patient treatment.

In one aspect, this disclosure is directed to a method of providing treatment to a patient. The method includes delivering a mixture to a targeted area within the patient. The mixture includes a therapeutic agent, a glue, and a radiopaque substance. In some cases, the therapeutic agent is a chemotherapeutic agent. In some cases, the glue is a cyanoacrylate. In some cases, the radiopaque substance is a lipid-based agent. In some cases, the mixture further includes at least one of a nanoparticle, an alcohol, an anesthetic agent, a radioactive material, a vasoconstricting agent, a vasodilating agent, an ultrasound contrast medium, a ultrasound microbubble, an anti-stromal agent, a pathway inhibitor, an immunotherapy agent, a vaccine, or a combination thereof. In some cases, the method also includes providing a needle for delivery of the mixture. In some cases, the needle defines an inner lumen comprising a coating. In some cases, the coating is at least one of an anticoagulant, a lipid-based agent, an acetone, or a nitromethane. In some cases, the method also includes providing a needle for delivery of the mixture. In some cases, the needle includes a first lumen and a second lumen. In some cases, the method also includes delivering the mixture through the first lumen, and delivering microcoils through the second lumen.

In another aspect, this disclosure is directed to a needle assembly for providing treatment to a patient. The needle assembly includes a needle housing defining a breakaway region, and a needle enclosed in the needle housing. The breakaway region exposes a portion of the needle. In some cases, the portion of the needle exposed by the breakaway region has a length to receive wire cutters. In some cases, when a force is applied on either side of the breakaway region, the portion of the needle exposed by the breakaway region breaks. In some cases, the needle includes multiple lumens. In some cases, the needle defines an inner lumen with a coating. In some cases, the coating is at least one of an anticoagulant, a lipid-based agent, an acetone, or a nitromethane. In some cases, the needle is configured to deliver a mixture to a targeted area of the patient. In some cases, the mixture includes a therapeutic agent, a glue, and a radiopaque substance. In some cases, the therapeutic agent is a chemotherapeutic agent. In some cases, the glue is a cyanoacrylate. In some cases, the radiopaque substance is a lipid-based agent. In some cases, the mixture further includes at least one of a nanoparticle, an alcohol, an anesthetic agent, a radioactive material, a vasoconstricting agent, a vasodilating agent, an ultrasound contrast medium, a ultrasound microbubble, an anti-stromal agent, a pathway inhibitor, an immunotherapy agent, a vaccine, or a combination thereof.

Particular embodiments of the subject matter described in this document can be implemented to realize one or more of the following advantages. First, the devices and methods provided herein can provide new systemic and targeted therapies impacting both tumor cells and the associated microenvironment and means of precision delivery are provided to improve local tumor control and to work in synergy with bespoke individualized therapies to customize patient care and outcome. Second, the chemotherapy diffusion pattern and extent for endoscopic ultrasound fine needle injection (EUS FNI) can abide by different principles than systemically delivered drugs. Third, local injection can allow delivery of greater drug concentrations and anti-stromal agents, thereby overcoming protective mechanisms developed by neoplastic cells and the microenvironment. Fourth, by increasing the intratumoral dose of chemotherapy or delivering a targeted agent based on the molecular landscape of the tumor and/or an anti-stromal agent, the therapeutic efficacy can be enhanced and the risk of adverse events can be reduced. Fifth, many patients, with a variety of tumors, such as those discussed above, can benefit from the local therapy. Sixth, many non-pancreatic solid and cystic lesions located within and remote to organs can benefit from local targeted therapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the invention are set forth in the accompanying drawings and the description below. Other features, objects, and advantages of the invention will be apparent from the description, drawings, and from the claims.

### DESCRIPTION OF DRAWINGS

FIG. 1 is a perspective view of a proximal portion of an endoscope with a needle assembly attached, in accordance with some embodiments provided herein.

FIG. 2 is a close-up perspective view of a proximal portion of an endoscope with a needle assembly attached, in accordance with some embodiments provided herein.

FIG. 3 is a perspective view of a distal portion of an endoscope with a needle assembly, in accordance with some embodiments provided here.

Like reference numbers represent corresponding parts throughout.

### DETAILED DESCRIPTION

This document describes methods and materials for improving cancer treatment. For example, this document describes methods and devices for local tumor control and customizable patient treatment.

Chemotherapy and radiation therapy have been used to treat pancreatic ductal adenocarcinoma (PDAC). However, the median survival expectancy for locally advanced pancreatic cancer (LAPC) remains at only 9-11 months, even with administration of chemoradiotherapy, and at only 6 months for patients with metastatic disease. Neoadjuvant therapy is routinely administered to patients with LAPC with the aim of tumor downstaging to potentially allow subsequent resection. The limited efficacy of conventional PDAC therapy results from the tumor biology, severe desmoplasia and altered tumor induced biophysical properties that result in neoplastic islands interspersed among dense stroma. This resulting tumor structure topography alters the pattern and limits chemotherapy penetrance to the tumor.

The devices and methods provided herein can provide new systemic and targeted therapies impacting both tumor cells and the associated microenvironment. In addition, means of precision delivery are provided to improve local tumor control that work in synergy with bespoke individualized therapies to customize patient care and outcome. The local injection can allow delivery of greater drug concentrations and anti-stromal agents, thereby overcoming protective mechanisms developed by neoplastic cells and the microenvironment. By increasing the intratumoral dose of chemotherapy or delivering a targeted agent based on the molecular landscape of the tumor and/or an anti-stromal agent, the therapeutic efficacy can be enhanced, and the risk of adverse events can be reduced.

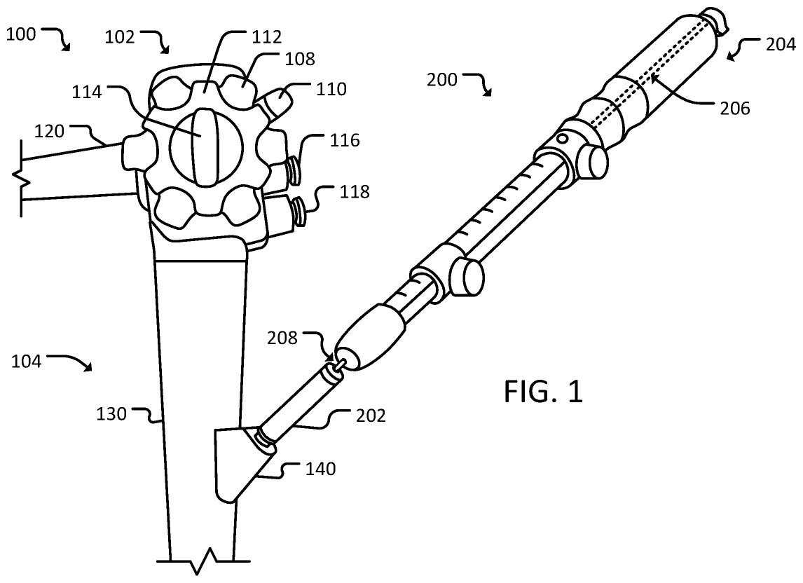

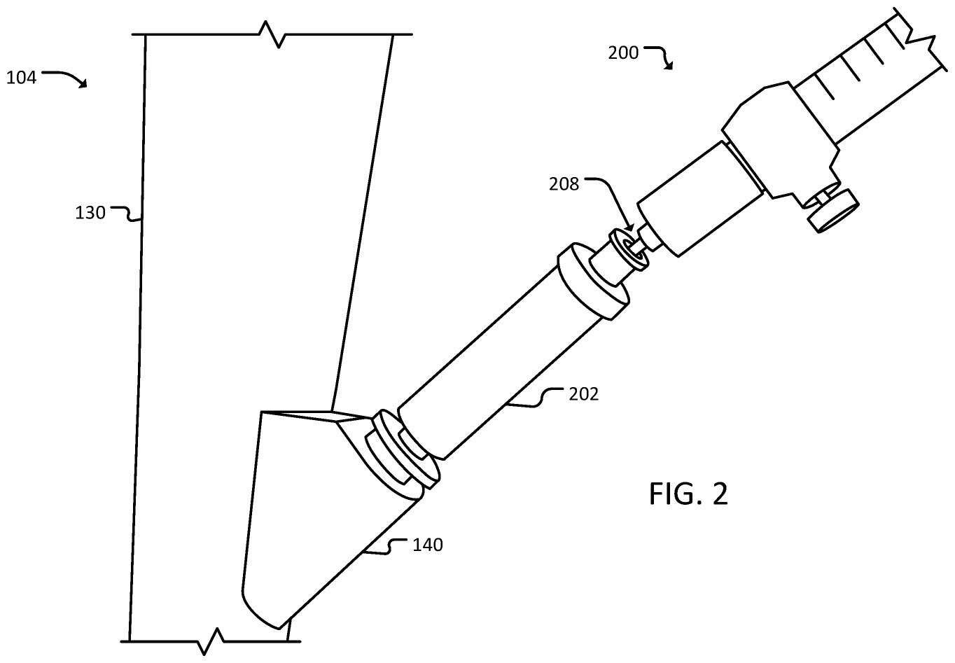

Referring to FIGS. 1-3, an example endoscope 100 can include a control head 102 and a body 104. In some cases, control head 102 can include an up/down knob 108, an up/down lock 110, a left/right knob 112, a left/right lock 114, a first button 116, a second button 118, and/or cable 120. In some cases, body 104 can include a body portion 130.

Up/down knob 108 can provide actuation of a distal portion (e.g., a bending portion) of endoscope 100 in a first plane. For example, up/down knob 108 can provide actuation in two directions within the first plane. Optionally, up/down knob 108 can provide actuation of the distal portion by mechanical means. For example, up/down knob 108 can provide actuation of the distal portion of the endoscope 100 by rotating a chain and sprocket on a gear that can be attached to angulation wires that extend to the distal portion of the endoscope 100. In some cases, the angulation wires can be attached to rings and pivot pins, such that rotation of the up/down knob 108 causes the distal portion to bend or move in a first direction and/or a second direction within the first plane. In another embodiment, up/down knob 108 can provide actuation of the distal portion by electrical means. For example, the up/down knob 108 can cause actuation of the distal portion via a motor.

Up/down lock 110 can prevent rotation of the up/down knob 108 when up/down lock 110 is engaged. In some cases, up/down lock 110 can cause previous rotation of the up/down knob 108, and therefore actuation of the distal portion of the endoscope 100, to be locked into place such that the position of the distal portion is maintained. Optionally, when up/down lock 110 is disengaged, up/down knob 108 can be rotated. Alternatively, when up/down lock 110 is disengaged, the position of up/down knob 108 can return to a neutral position (e.g., an original or starting position, such as a position that causes no bending of the distal end of endoscope 100).

Left/right knob 112 can provide actuation of a distal portion (e.g., a bending portion) of endoscope 100 in a second plane, different from the first plane, for example, in two directions within the second plane. Optionally, left/right knob 112 can provide actuation of the distal portion of the endoscope 100 by rotating a chain and sprocket on a gear that can be attached to angulation wires that extend to the distal portion of the endoscope 100. In some cases, the angulation wires can be attached to rings and pivot pins, such that rotation of the left/right knob 112 causes the distal portion to bend or move in a first direction and/or a second direction within the second plane. Alternatively, left/right knob 112 can provide actuation of the distal portion by electrical means. For example, the left/right knob 112 can cause actuation of the distal portion via a motor.

Left/right lock 114 can prevent rotation of the left/right knob 112 when left/right lock 114 is engaged. In some cases, left/right lock 114 can cause previous rotation of the left/right knob 112, and therefore actuation of the distal portion of the endoscope 100, to be locked into place such that the position of the distal portion is maintained. In some embodiments, when left/right lock 114 is disengaged, left/right knob 112 can be rotated. Alternatively, when left/right lock 114 is disengaged, the position of left/right knob 112 can return to a neutral position (e.g., an original or starting position, such as a position that causes no bending of the distal end of the endoscope 100).

First button 116 and/or second button 118 can be valves. For example, first button 116 and/or second button 118 can be a suction valve, an air valve, a water valve, an anti-reflux valve. In some cases, first button 116 and/or second button 118 can include a vent hole. Optionally, first button 116 and/or second button 118 can be remote switches. For example, first button 116 and/or second button 118 can control a light (e.g., turning the light on and/or off), a camera (e.g., turning the camera on and/or off), or a similar device or feature. In some cases, first button 116 and/or second button 118 can be removable, for example, for cleaning. In some cases, first button 116 and/or second button 118 can be electronic switches that can control valves, pumps, or other devices of the endoscope 100. The control valves can interact with various channels of the endoscope 100. Optionally, the control valves can interact with various channels of the endoscope 100 without coming into contact with the fluids (e.g., gases and/or liquids) inside the channels. For example, a mechanical pump can surround the channel and vary a pressure around the channel to cause pumping.

Cable 120 can be coupled to the other portions of control head 102 of endoscope 100 and can provide means of connection to an external electronic device, such as an endoscopy tower (not shown). The endoscopy tower can include an electronic control valve, such as an air control valve and/or a water control valve. In some cases, the endoscopy tower can include a video image processor that can receive video information from a camera located at a distal end of the endoscope 100. Optionally, the endoscopy tower can provide imaging, such that a user can view images and/or video via the camera. The endoscopy tower can also allow a user to modify parameters of the video or image, such as contrast, brightness, zoom, etc.

In some cases, body portion 130 can taper, or decrease in diameter as body portion 130 extends away from control head 102. Body portion 130 can provide additional features and/or components of endoscope. For example, body portion 130 can include a valve 140 that can provide access from body portion 130 to a distal end of endoscope 100. In some cases, valve 140 can be positioned at an angle from the body portion 130 such that inserting a tool into valve 140 can be made easier based on the angle between the valve 140 and the body portion 130. Optionally, valve 140 can include a cap, or deflectable valve, such that when a biopsy tool is not being used, valve 140 remains closed to reduce the chance of infection, cross contamination, or spread of other bacteria. In some cases, valve 140 can include means for securing or attaching a tool to valve 140, and accordingly, body portion 130. For example, valve 140 can be a luer lock.

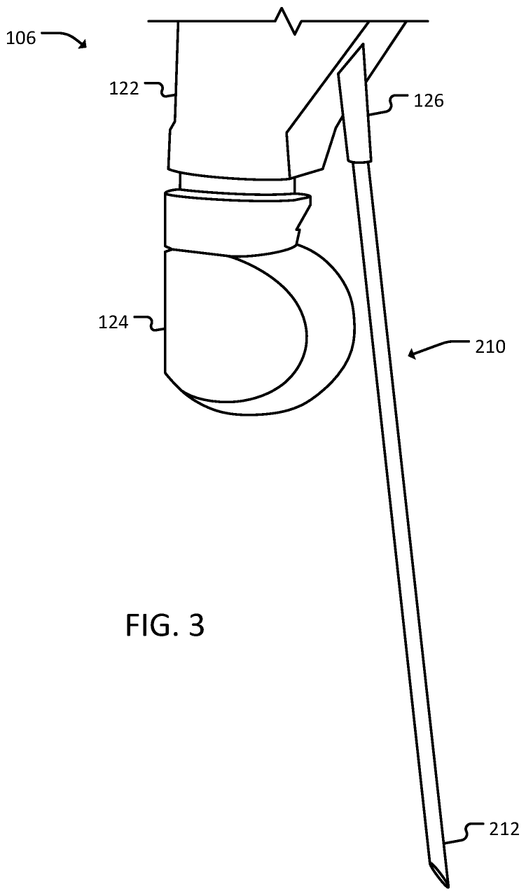

In some cases, body portion 130 can include a tube 106 (shown in FIG. 3) that can be inserted into a patient. In some cases, tube 106 can be made of metal and/or plastic. In some cases, tube 106 can include a bending section. In some cases, the bending section can be located at a distal portion of tube 106. In some cases, tube 106 can be deformable. In some cases, tube 106 can provide access from a proximal portion (e.g., control head 102 and/or body 104) to a distal end of the endoscope 100. In some cases, tube 106 can provide access for control elements, channels, and/or other components of endoscope 100.

Tube 106 can include a distal portion 122. Distal portion 122 can include an ultrasound head 124. Ultrasound head 124 can include a transducer (not shown). In some cases, the transducer can be a linear transducer such that ultrasound images captured are parallel to endoscope 100. In some cases, the transducer can be a radial transducer such that ultrasound images captured are perpendicular to endoscope 100. In some cases, the transducer can be front viewing at the tip (rather than side) of endoscope 100. In some cases, ultrasound head 124 can include a camera such that a user can direct endoscope 100 to a desired location. In some cases, ultrasound head 124 can include a light source to aid in directing endoscope 100 to a desired location. In some cases, ultrasound head 124 can include other devices/features such as apertures, channels, or nozzles.

As shown in FIGS. 1-3, the valve 140 can receive a needle assembly 200. Needle assembly 200 can include a needle housing 204 and a needle 206. Needle housing 204 can include a valve coupling region 202 and a breakaway region 208.

In some cases, needle 206 can include a coating along an inner lumen of needle 206. For example, the inner lumen can be coated with an anticoagulant (e.g., to reduce the risk of clot formation), a lipid-based agent or other fatty solvent (e.g., to reduce polymerization of a glue and prevent occlusion of the inner lumen of needle 206 by the glue), a acetone or nitromethane (e.g., to reduce polymerization of a glue and prevent occlusion of the inner lumen of needle 206 by the glue), or a combination thereof.

In some cases, the instrument channel can be coated with an agent. For example, the instrument channel can be coated with an anticoagulant (e.g., to reduce the risk of clot formation), a lipid-based agent or other fatty solvent (e.g., to reduce polymerization of a glue and prevent occlusion of the instrument channel by the glue), an acetone or nitromethane (e.g., to reduce polymerization of a glue and prevent occlusion of the instrument channel by the glue), or a combination thereof.

In some cases, needle 206 can include multiple lumens (e.g., two or three lumens). In some cases, multiple lumens can be used such that different agents, in different lumens, are injected at different injection rates. In some cases, multiple lumens can inject different agents at different times, allowing for varying exposure times. In some cases, multiple lumens can be used to deliver a first inactive agent and a second inactive agent separately, such that the combination of the first inactive agent and the second inactive agent causes activation of the agents. In some cases, a final injection of a liquid or physical sealant can be used to prevent retrograde diffusion of the injected agent along the needle track.

In some cases, multiple lumens can be used to inject microcoils through a first lumen and a liquid agent through a second lumen, eliminating the need to remove needle 206 and insert a second needle. Optionally, the microcoils can be embedded with various agents. For example, the microcoils can be embedded with a glue, a lipid-based agent, a hemostatic agent, a chemotherapeutic agent, a radiosensitive agent, an anti-stromal agent, or a combination thereof.

In some cases, needle 206 can have an outer diameter that is sufficiently large enough to occupy substantially all the space within the instrument channel. Such a diameter can reduce reflux of blood clots, glue, or other materials which could damage endoscope 100, and can negatively impact imaging. In some cases, needle 206 can have an increased diameter by using an inflatable balloon that can be inflated at a distal portion of the instrument channel. In addition, the inflatable balloon can aid in maintaining needle 206 in a center of the instrument channel, and in a correct plane. In some cases, needle 206 can have an increased diameter by using a thicker outer sheath for needle 206. In some cases, needle 206 can have an increased diameter by using struts.

In some cases, needle tip 212 can include an inflatable balloon. For example, the inflatable balloon can be inflated once needle tip 212 is advanced into the vessel or duct (e.g., bile duct or pancreatic duct), or fluid collection to help safely hold needle 206 in place. In some cases, the inflatable balloon can be configured such that when inflated, the inflatable balloon overlies needle tip 212, to provide increased safety, for example.

Breakaway region 208 can provide an exposed region of needle 206. In some cases, breakaway region 208 allows needle 206 to be cut or broken at a proximal end of needle 206, and a proximal end of endoscope 100. In some cases, breakaway region 208 can be sized to allow a wire cutter to reach needle 206, such that needle 206 can be cut. In some cases, breakaway region 208 can be sized to prevent a wire cutter from reaching needle 206. In some cases, needle 206 can be broken by applying force on a proximal region of needle assembly 200 and valve coupling region 202. By cutting or breaking needle 206 at a proximal portion of needle 206, an amount of glue, agents, or other fluids that are retracted into the instrument channel can be reduced, while increasing safety of a user by reducing the risk of a needle stick. For example, needle tip 212 can be retracted into the instrument channel, or into a needle sheath inside the instrument channel, such that needle tip 212 is no longer exposed distal to the instrument channel. Endoscope 100 can be removed from the patient, with needle 206 being removed as well. Once removed, needle 206 can be cut or broken at breakaway region 208, such that distal portion 210 and needle tip 212 can be removed from a distal portion of the instrument channel, while a proximal portion of needle 206 can be removed from valve 140. Such a removal process can reduce an amount of glue, agents, or other fluids from entering, and potentially occluding, the instrument channel. In some cases, breakaway region 208 can also include means for securing needle 206. For example, breakaway region 208 can have an adapter configured to secure needle 206 such that needle tip 212 protrudes from the instrument channel a desired amount, or such that needle tip 212 is securely within the instrument channel.

While the devices and methods herein have been described with respect to an endoscope, various other modalities may be used to administer therapy. For example, endoscopic ultrasound, standard endoscopy, interventional radiology guided delivery, and via percutaneous routes.

Further, in some cases, the devices and methods described herein can be used to perform and provide additional treatments. For example, endoscopic ultrasound delivery of an inferior vena cava filter to inhibit clot embolization can be performed. As another example, endoscopic ultrasound delivery of vascular stents can be performed.

### Delivery Agent

In some cases, a delivery agent may be used in conjunction with the needle assembly 200. For example, the therapeutic agent that is being administered to the patient may include a glue and/or a lipid-based agent, or other radiopaque liquid substance. In some cases, the glue is a cyanoacrylate (e.g., Dermabond). In some cases, the therapeutic agent may be a chemotherapeutic. Some advantages of using a glue and/or a radiopaque liquid substance as a delivery agent are as follows. First, the viscosity of the delivery compound delivered can be easily altered to obtain an ideal viscosity. Second, the compound can allow diffusion of the therapeutic agent throughout solid and cystic lesions. Third, imaging modalities (e.g., fluoroscopy, endoscopic ultrasound, etc.) can be used to easily observe a pattern and extent of spread of the therapeutic agent. Fourth, the compound, and therefore the therapeutic agent, can remain within the target site for prolonged periods of time, allowing for increased durability of the treatment. Fifth, a collateral benefit can include vascular compromise with diminished blood flow and cellular death (e.g., of cancerous cells). In some cases, the glue, lipid-based agent, and/or therapeutic agent compound can also be combined with nanoparticles, alcohol, anesthetic agents, radioactive materials, vasoconstriction agents, vasodilating agents, ultrasound contrasts (e.g., to aid with visualization), ultrasound microbubbles (e.g., containing other agents described herein), anti-stromal agents (e.g., to break down fibrosis), targeted therapy based on a molecular signature of the tumor (e.g., mammalian target of rapamycin (mTOR) or poly ADP ribose polymerase (PARP) pathway inhibitors), immunotherapy agents, a vaccine, or a combination thereof. In some cases, an injectant can also serve as a radiopaque, possibly biodegradable, liquid fiducial to facilitate proton and photon radiation therapy.

### Target Lesions

Numerous targets, pathologies, and therapies can benefit from the devices and methods described herein. For example, solid pancreatic tumors may include PDAC, functioning and non-functioning neuroendocrine tumors, lymphoma, second metastasis to the pancreas, etc. There are also several cystic pancreatic tumors (CPT) such as intraductal papillary mucinous neoplasia, mucinous cystic neoplasia, and serous cystadenomas, among others, that would benefit from local therapy. Similarly, many non-pancreatic solid and cystic lesions located within and remote to organs may benefit from local targeted therapy. Targets also include various types of bleeding lesions such as varices (esophageal, gastric, anastomotic, etc.), ulcers (gastroduodenal, etc.), pseudoaneurysms (splenic artery, etc.), etc.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described herein as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures does not necessarily require the particular order shown, or sequential order, to achieve desirable results. In certain implementations, multitasking and parallel processing may be advantageous.

1. A method of providing treatment to a patient, the method comprising:
delivering a mixture to a targeted area of the patient, the mixture comprising:
a therapeutic agent;
a glue; and
a radiopaque substance.

2. The method of claim 1, wherein the therapeutic agent is at least one of a chemotherapeutic agent, an immunomodulatory agent, or a vaccine.

3. The method of claim 1, wherein the glue is a cyanoacrylate.

4. The method of claim 1, wherein the radiopaque substance is a lipid-based agent.

5. The method of claim 1, wherein the mixture further comprises at least one of a nanoparticle, an alcohol, an anesthetic agent, a radioactive material, a vasoconstricting agent, a vasodilating agent, an ultrasound contrast medium, a ultrasound microbubble, an anti-stromal agent, a pathway inhibitor, an immunotherapy agent, a vaccine, or a combination thereof.

6. The method of claim 1, further comprising providing a needle for delivery of the mixture, wherein the needle defines an inner lumen comprising a coating.

7. The method of claim 6, wherein the coating is at least one of an anticoagulant, a lipid-based agent, an acetone, or a nitromethane.

8. The method of claim 1, further comprising providing a needle for delivery of the mixture, wherein the needle comprising a first lumen and a second lumen.

9. The method of claim 8, further comprising delivering the mixture through the first lumen, and delivering microcoils through the second lumen.

10. A needle assembly for providing treatment to a patient, the needle assembly comprising:
a needle housing defining a breakaway region; and
a needle enclosed in the needle housing,
wherein the breakaway region exposes a portion of the needle.

11. The needle assembly of claim 10, wherein the portion of the needle exposed by the breakaway region has a length to receive wire cutters.

12. The needle assembly of claim 10, wherein when force is applied on either side of the breakaway region, the portion of the needle exposed by the breakaway region breaks.

13. The needle assembly of claim 10, wherein the needle comprises multiple lumens.

14. The needle assembly of claim 10, wherein the needle defines an inner lumen comprising a coating.

15. The needle assembly of claim 14, wherein the coating is at least one of an anticoagulant, a lipid-based agent, an acetone, or a nitromethane.

16. The needle assembly of claim 10, wherein the needle is configured to deliver a mixture to a targeted area of the patient, the mixture comprising:
a therapeutic agent;
a glue; and
a radiopaque substance.

17. The needle assembly of claim 16, wherein the therapeutic agent is a chemotherapeutic agent.

18. The needle assembly of claim 16, wherein the glue is a cyanoacrylate.

19. The needle assembly of claim 16, wherein the radiopaque substance is a lipid-based agent.

20. The needle assembly of claim 16, wherein the mixture further comprises at least one of a nanoparticle, an alcohol, an anesthetic agent, a radioactive material, a vasoconstricting agent, a vasodilating agent, an ultrasound contrast medium, a ultrasound microbubble, an anti-stromal agent, a pathway inhibitor, an immunotherapy agent, a vaccine, or a combination thereof.
